(19) United States
(12) Patent Application Publication
(10) Pub. No.: US 2021/0023359 A1
(43) Pub. Date: Jan. 28, 2021

(54) OVERTUBE

(71) Applicant: OLYMPUS CORPORATION, Tokyo (JP)

(72) Inventor: Hiroyuki KOJO, Kokubunji-shi (JP)

(73) Assignee: OLYMPUS CORPORATION, Tokyo (JP)

(21) Appl. No.: 17/060,188

(22) Filed: Oct. 1, 2020

Related U.S. Application Data
(63) Continuation of application No. PCT/JP2018/014726, filed on Apr. 6, 2018.

(51) Int. Cl. A61M 39/10; A61B 1/01; A61B 1/00; A61B 1/018; A61B 1/233; A61M 25/09

(52) U.S. Cl. CPC A61M 39/105; A61B 1/01; A61B 1/00135; A61B 1/018; A61M 2210/0618; A61M 25/09; A61M 2025/091; A61M 2205/02; A61B 1/233

(57) ABSTRACT

An embodiment provides an overtube which includes: a plurality of lumens including a lumen with a pliable folded structure at a portion to be inserted into a treatment target; and a grip portion having a rigidity in a radial direction, being easily inserted into the treatment target, allowing a medical device to be easily inserted thereinto, and facilitating administration of a drug.

### CROSS-REFERENCE TO RELATED APPLICATIONS

This application is a Continuation Application of PCT Application No. PCT/JP2018/014726, filed Apr. 6, 2018, the entire contents of which are incorporated herein by reference.

### BACKGROUND OF THE INVENTION

### 1. Field of the Invention

The present invention relates to an overtube comprising a lumen with a folded structure.

### 2. Description of the Related Art

A medical overtube used for insertion of a medical device into a body cavity or a surgical portal is generally known. If such an overtube is configured to include a plurality of insertion paths (lumens), a plurality of medical devices such as forceps and a catheter, as well as an endoscope, can be simultaneously inserted as insertion objects. Since an overtube with a plurality of lumens increases in a cross-sectional area when inflated, various schemes have been proposed to facilitate insertion, such as reducing the cross-sectional area by providing a pliability that allows for collapse, for example. A flexible overtube formed of a flexible material such as a resin film or a synthetic film is proposed in, for example, Patent Document 1 (Japanese Patent No. 5213380).

In the above-described flexible overtube, it is preferable that a distal side to be inserted into the body cavity have a high flexibility, that is, a high pliability. On the other hand, it is preferable that a grip portion to be held by the operator have a low flexibility, that is, a high rigidity (stiffness), to achieve stable operation. There is also a generally pliable overtube including, for example, a lumen into which an endoscope is inserted, and a lumen into which a catheter for drug administration is inserted. Since such an overtube is configured in such a manner that the entire overtube including the endoscope to be inserted is held by the operator's hand, the pliability of the holding portion causes a lumen other than the lumen into which the endoscope is to be inserted to be collapsed. The collapse of the other lumen may possibly prevent insertion of a catheter to be introduced later, or stop the flow of a drug to restrict its administration.

### BRIEF SUMMARY OF THE INVENTION

According to an embodiment of the present invention, there is provided an overtube in which at least two lumens are aligned and which is inserted into a subject, said at least two lumens including a first lumen and a second lumen, the overtube comprising: a first wall portion which forms an anterior side of the second lumen to receive insertion, the anterior side having a folded structure in which the distal side is folded so as to be unfoldable in a radial direction; and a second wall portion which forms a posterior side of the second lumen to be gripped and which is more rigid than the first wall portion in the radial direction, the posterior side including an opening of the second lumen.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram showing an external configuration of an overtube according to a first embodiment of the present invention.

FIG. 2A is a diagram showing a structure of an A-A cross section at an anterior portion of the overtube shown in FIG. 1.

FIG. 2B is a diagram showing a structure of a B-B cross section at a central portion of the overtube shown in FIG. 1.

FIG. 2C is a diagram showing a structure of a C-C cross section at a posterior portion of the overtube shown in FIG. 1.

FIG. 3A is a diagram showing a state in which an endoscope and a catheter prior to infusion of a drug are inserted into an overtube.

FIG. 3B is a diagram showing a structure of a cross section of the distal side of the overtube that is inserted into the paranasal sinuses FIG. 4 is a conceptual diagram showing a state in which the overtube is inserted into the paranasal sinuses through the nasal passage.

FIG. 5A is a diagram showing a state in which an endoscope and a catheter during infusion of a drug are inserted into an overtube.

FIG. 5B is a diagram showing a cross-sectional structure of a distal side of the overtube that is inserted into the paranasal sinuses.

FIG. 6 is a diagram showing a first modification of a folded structure of a wall portion.

FIG. 7 is a diagram showing a second modification of the folded structure of the wall portion.

FIG. 8A is a diagram showing a cross-sectional configuration of a state in which a catheter including a balloon is inserted into the overtube.

FIG. 8B is conceptual diagram showing a state in which the balloon in the overtube is inflated.

FIG. 9A is a conceptual diagram showing a state in which an overtube into which a catheter including a balloon has been inserted is inserted into a frontal sinus.

FIG. 9B is a conceptual diagram showing a state in which the balloon of the catheter is inflated.

FIG. 9C is a diagram showing a state in which a drug is administered into the sinus through the catheter.

FIG. 9D is a diagram showing a state in which the drug administered into the sinus is discharged by suction, with a requisite amount of the drug maintained.

FIG. 10A is a conceptual diagram showing a state in which a catheter including a balloon is inserted into a frontal sinus so as to protrude from an overtube in a third example of drug administration.

FIG. 10B is a conceptual diagram showing a state in which the balloon of the catheter is inflated.

FIG. 10C is a diagram showing a state in which a drug is administered into the sinus through the catheter.

FIG. 10D is a diagram showing a state in which the drug is discharged by suction from the sinus through the catheter.

### DETAILED DESCRIPTION

Hereinafter, embodiments of the present invention will be described in detail with reference to the drawings.

An embodiment according to the present invention provides an overtube which includes: a plurality of lumens including a lumen with a pliable folded structure at a portion to be inserted into a treatment target; and a grip portion having a rigidity in a radial direction, being easily inserted into the treatment target, allowing a medical device to be easily inserted thereinto, and facilitating administration of a drug.

### First Embodiment

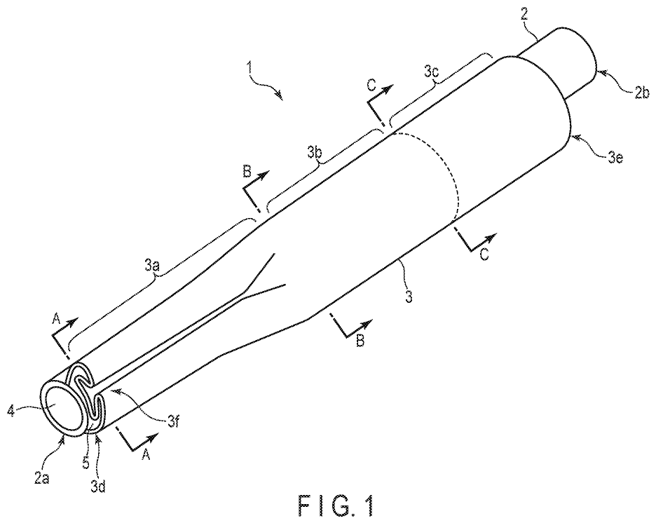

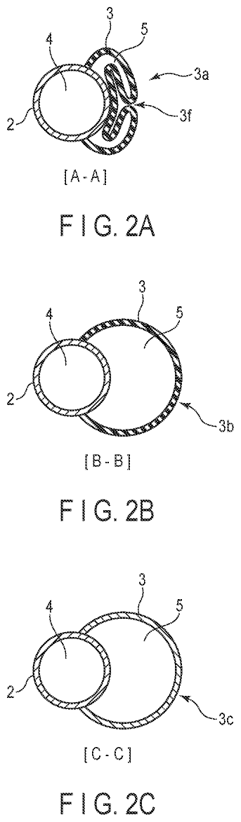

An overtube 1 according to a first embodiment will be described. FIG. 1 is a diagram showing an external configuration of an overtube according to a first embodiment. FIG. 2A is a diagram showing a structure of an A-A cross section at an anterior portion of the overtube shown in FIG. 1, FIG. 2B is a diagram showing a structure of a B-B cross section at a central portion of the overtube, and FIG. 2C is a diagram showing a structure of a C-C cross section at a posterior portion of the overtube. The overtube 1 to be described below includes at least two insertion paths (hereinafter referred to as “lumens”) into which medical insertion objects are respectively inserted. Three or more lumens may be provided, and some of them may have a folded structure (to be described later), even though such a configuration will not be described in the present embodiment. Let us assume that the medical insertion objects are medical devices including a flexible endoscope (hereinafter referred to as an “endoscope”), a guide wire, a catheter, a sheath, or the like, and a treatment tool such as forceps. In the description that follows, a soft endoscope will be described as an example; however, a rigid endoscope may be used depending on an observation target and a treatment target.

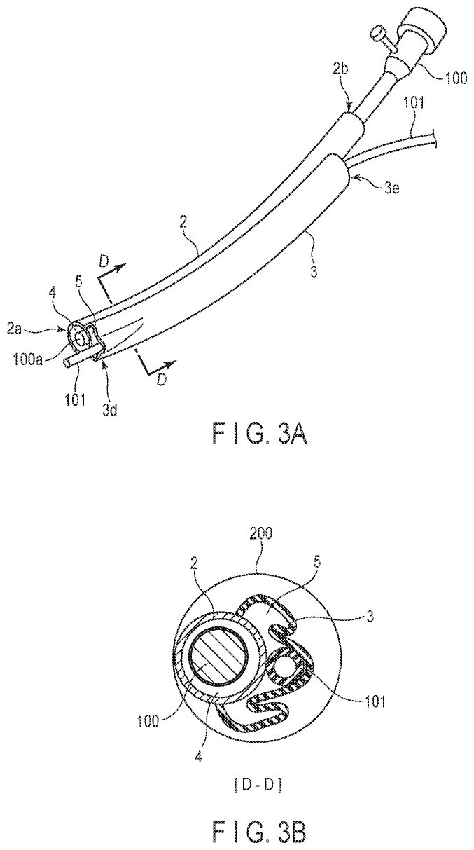

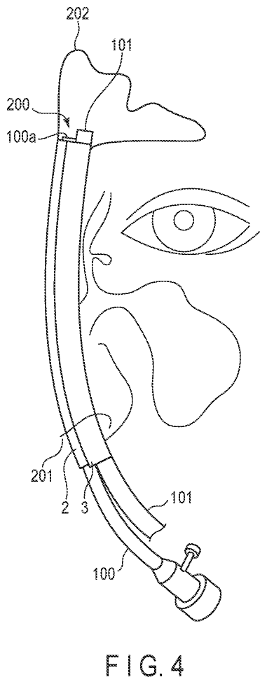

The overtube 1 shown in FIG. 1 includes two adjacent lumens 4 and 5 formed by wall portions. As shown in FIG. 3A, the lumen 4 (first lumen) of the overtube 1 includes a wall portion formed of a primary tube 2, and an insertion portion of an endoscope 100 is inserted thereinto. The lumen 5 (second lumen) adjacent thereto includes a wall portion formed of a secondary tube 3, and medical devices including, for example, a catheter 101 (to be described later) and a treatment tool such as forceps are inserted thereinto. Of these, the primary tube 2 is formed using, for example, a resin material so as to maintain a substantially cylindrical shape in the radial direction and to have a flexibility that allows bending in a given direction as viewed in the longitudinal axis direction. The secondary tube 3 is provided in such a manner that its portion in the longitudinal axis direction shares an outer surface of the primary tube 2 as a wall portion, and has a C shape (crescent shape) when a folded portion of the wall portion is unfolded. As a matter of course, a configuration may be adopted in which two separately formed tubes are aligned in the longitudinal axis direction and coupled. The primary tube 2 and the secondary tube 3 may have the same length or different lengths.

The wall portion of the secondary tube 3 is formed in such a manner that three portions with different characteristics, that is, a distal section 3a (first wall portion) on an anterior side to be inserted, a central section 3b (third wall portion), and a proximal section 3c (second wall portion) on a posterior side to be gripped by the operator, are continuously and integrally formed from the distal side as viewed in the longitudinal axis direction.

As shown in FIGS. 1 and 2A, the distal section 3a of the secondary tube 3 has a folded structure 3f in which the wall portion is folded toward the inner surface side when no insertion object is inserted into the lumen 5. As shown in FIG. 2B, the secondary tube 3, namely, the lumen 5, is formed in such a manner that both of its ends rise from positions apart from each other on the outer peripheral surface of the primary tube 2 to describe an arc. That is, the lumen 5 shares a portion of the outer peripheral surface of the primary tube 2 as a wall portion of the lumen 5 as viewed in the longitudinal axis direction. The wall portion with the folded structure 3f is folded along the outer peripheral surface of the primary tube (wall portion) 2 which forms the lumen 4.

The folded structure 3f at the distal section 3a has a length that is at least equal to or greater than a distance to which it is to be inserted into the patient to receive insertion (e.g., from the nasal cavity to the ventilation hole (orifice) of the paranasal sinuses). The distal section 3a is formed of a resin material or an elastic member such as rubber, and has a pliability and a stretchability. When an insertion object is inserted into the distal section 3a, the folded portion is unfolded to expand in accordance with the cross-sectional shape and the cross-sectional size of the insertion object. When maximally unfolded, the folded structure 3f at the distal section 3a takes, for example, a C-shape (crescent shape) in which a part of a circle is missing, as shown in FIG. 2B. Also, the distal section 3a may be formed, when unfolded, to have a tapered shape that spreads toward the distal end in such a manner that a cross-sectional area of the distal section 3a becomes larger than a cross-sectional area of the central section 3b shown in FIG. 2B. As a matter of course, the shape of the folded structure 3f at the distal section 3a when maximally unfolded is not limited to a circular shape, and may expand to an elliptical or rectangular shape depending on the application (e.g., the shape of the insertion object to be inserted).

Accordingly, as shown in FIG. 2A, the distal section 3a of the overtube 1 of the present embodiment is inserted into the body cavity of the observation target or the surgical portal, with the folded structure 3f folded. By the folding, the cross-sectional area can be reduced, thus facilitating the insertion operation and increasing the insertability. As shown in FIG. 2C, the proximal section 3c of the secondary tube 3 does not include a wall portion with a fold-in structure, maintains the same shape as the C-shape in the unfolded state, and an insertion port 3e is bored on the proximal side. The proximal section 3c has a rigidity (stiffness) that allows the cross-sectional shape to be maintained without being substantially deformed in the radial direction even when gripped by a hand of the operator, etc. The proximal section 3c is formed of, for example, a resin material, a metal material, or a combination thereof. If the fold-in structure of the wall portion is provided in an overtube 1 that is not to be reused, the wall portion need not necessarily return to the same folded shape after use.

The central section 3b of the secondary tube 3 is a switching portion that couples the distal section 3a and the proximal section 3c to smoothly change the difference in rigidity. The difference in rigidity need not be changed smoothly (linearly), and may be switched stepwise. The central section 3b does not have a folded structure and includes a wall portion that maintains a C-shape when unfolded, as shown in FIG. 2B, but has a rigidity that allows the cross-sectional shape to be deformed to a certain degree when gripped by a hand of the operator, etc. The central section 3b may be formed using a material equivalent to the material forming the distal section 3a to increase the thickness and ensure the rigidity.

The three sections of the secondary tube 3 of the present embodiment only need to have different rigidity characteristics related to the presence or absence of the above-described deformation, and may be formed of either the same material or different materials. When the three sections are formed of the same resin material, they can be formed by integral molding by varying the thickness so as to increase from the distal side to the proximal side. Alternatively, the three sections may be formed only of resin materials, using resin materials with different hardnesses.

Furthermore, when the three sections are formed using different materials, for example, when the distal section 3a and the central section 3b are formed of a resin material and the proximal section 3c is formed of a metal material, as shown in FIG. 2C, they can be formed by two-color molding technology. Alternatively, the distal section 3a and the central section 3b may be formed of resin materials with different hardnesses, and the proximal section 3c may be formed of a composite material obtained by combining a resin material and a metal material. In this manner, each section can be formed by appropriately combining the formation materials.

[First Example of Drug Administration]

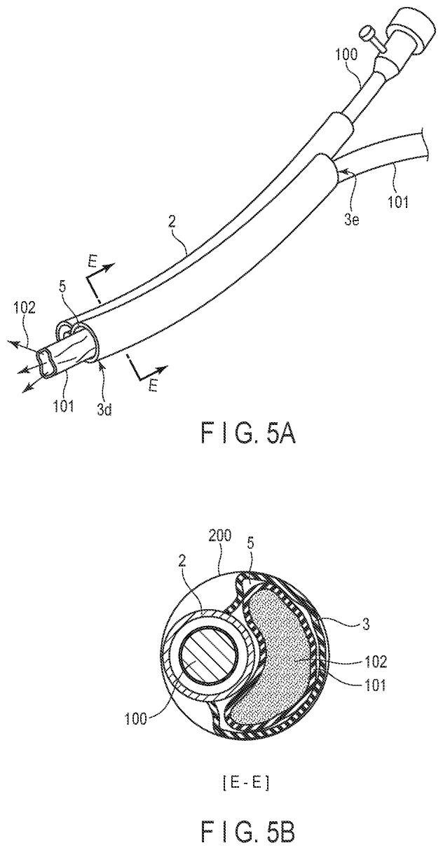

A first example will be described in which an endoscope and a medication catheter (or a medication sheath) are inserted into a frontal sinus 202 of the paranasal sinuses through the two lumens 4, 5 of the overtube 1 of the present embodiment. FIG. 3A is a diagram showing a state in which the endoscope 100 and the catheter 101 prior to infusion of a drug are inserted into the overtube 1, and FIG. 3B is a diagram showing a structure of a cross section (a D-D cross section in FIG. 3A) of the distal side of the overtube 1 that is inserted into the paranasal sinuses. FIG. 4 is a conceptual diagram showing a state in which the overtube 1 is inserted into the paranasal sinuses (frontal sinus 202) through the nasal passage. FIG. 5A is a diagram showing a state in which the endoscope 100 and the catheter 101 during infusion of a drug are inserted into the overtube 1, and FIG. 5B is a diagram showing a structure of a cross section (E-E cross section in FIG. 5A) of the distal side of the overtube 1 when inserted into the paranasal sinuses.

As shown in FIG. 3A, an insertion portion of the endoscope 100 is introduced from the proximal-side opening 2b of the lumen 4 of the overtube 1, and the distal portion 100a of the insertion portion provided with an observation window is inserted up to the opening 2a at the distal end of the tube. After that, as shown in FIG. 4, a distal end of the overtube 1 to which the endoscope 100 is attached is inserted from the nasal cavity 201 through the nasal passage to reach an orifice 200 of the frontal sinus 202. At this time, the operator pinches the distal section 3a of the secondary tube 3 with his or her fingers, and gradually introduces it from the nasal cavity while viewing an observation image by the endoscope 100. The insertion is stopped when the distal portion 100a of the endoscope 100 reaches the orifice 200 of the frontal sinus 202. After the stop, the proximal section 3c of the overtube 1 is gripped to support the endoscope 100.

Thereafter, a distal portion of a catheter 101 for drug administration is introduced from the insertion port 3e of the secondary tube 3 up to the distal opening 3d. In general, when the operator grips the overtube 1 to support the endoscope 100, insertion of a treatment tool such as the catheter 101 is performed by an assistant or the like. The catheter 101 of the present embodiment has a stretchability that allows expansion and an increase in diameter in accordance with the amount and viscosity of the drug administered, or the drag generated (difficulty to flow).

FIG. 3B is a cross-sectional diagram of the orifice 200 of the frontal sinus 202, with the endoscope 100 inserted into the lumen 4 of the overtube 1, and the catheter 101 inserted into the lumen 5. At this time, the wall portion of the fold-in structure of the lumen 5 is still in a folded state, and bulges only by an amount corresponding to the diameter of the catheter 101. As shown in FIG. 5A, a gel-like drug 102 is injected from the proximal side (not illustrated) of the catheter 101. As the injected drug 102 flows in, the catheter 101 is expanded in the radial direction and the folded secondary tube 3 is unfolded, as shown in FIG. 5B, thereby causing the drug 102 to flow from the opening of the secondary tube 3 into the frontal sinus 202 and be administered thereto. At this time, when the folded structure of the secondary tube 3 has a cross section larger than the size (opening diameter) of the orifice 200 of the frontal sinus 202, the unfolded-state shape is restricted due to the influence of the shape of the orifice 200. FIG. 5B shows an example of fan-shaped unfolding.

Since the viscosity of a gel-like drug is higher than that of a liquid such as water, it requires more time for such a drug to flow from the proximal side to the distal side under the effect of the drag. Therefore, in order to shorten the time required for administration of the drug 102, a catheter 101 into which a certain amount of the drug 102 is filled in advance is inserted into the lumen 5. Furthermore, the catheter 101 may be formed using a shape that expands stepwise or expands in a tapered shape from the proximal side to the distal side when inflated, so as to reduce the drag against the flow. Even though the drug 102 is administered using the catheter 101 in the present embodiment, a drug infuser such as a syringe (not illustrated) may be directly introduced into the lumen 5 of the secondary tube 3 to directly administer the drug 102 through the lumen 5.

As described above, the overtube 1 of the present embodiment is configured, at the distal section 3a to be inserted into the body cavity or the surgical portal, to have the folded structure 3f as the wall portion forming a lumen other than the lumen 4 into which the endoscope is inserted. The folded structure 3f can reduce the cross-sectional area during insertion, thus facilitating the insertion operation with the overtube 1. When a treatment tool is inserted into the other lumen 5 or a drug is administered thereto, the folded secondary tube 3 is unfolded, and the diameter of the lumen 5 can be increased to a desired cross-sectional size, thereby facilitating insertion of a medical device and outflow of the drug.

Furthermore, since the operator grips the proximal section 3c on the proximal side having a rigidity that does not cause deformation in the radial direction, and holds and operates the endoscope 100, etc. after the insertion of the overtube 1 is completed, a stable operation can be realized. In the secondary tube 3 of the overtube 1, a central section 3b having a certain degree of pliability is provided between the rigid proximal section 3c and the pliable distal section 3a, while maintaining an outer shape (unfolded shape). The central section 3b allows smooth switching from the pliability to the rigidity. This prevents buckling (bending) from occurring between the proximal section 3c and the distal section 3a due to the step difference of the rigidity value, thus ensuring the operability of the overtube 1. Since such buckling does not occur, it is possible to prevent the failure of catheter feeding.

[First Modification of Folded Structure]

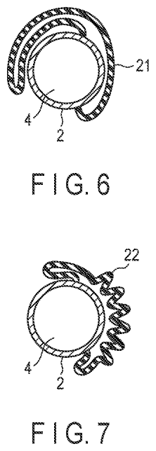

Next, a first modification of the folded structure of the wall portion at the distal section 3a of the secondary tube 3 of the overtube 1 will be described with reference to FIG. 6.

The secondary tube 21 has a structure in which the wall portion is folded to be wound around an outer peripheral surface of the primary tube 2. With this folded structure, it is possible to reduce the cross-sectional area during insertion, thus facilitating the insertion operation and increasing the insertability at the time of insertion into the body cavity of the observation target or a surgical portal.

[Second Modification of Folded Structure]

Next, a second modification of the folded structure of the wall portion at the distal section 3a of the secondary tube 3 of the overtube 1 will be described with reference to FIG. 7.

The secondary tube 22 has a structure in which the wall portion is accordion-folded along an outer peripheral surface of the primary tube 2. With this folded structure, it is possible to easily unfold the accordion-folded wall portion when a treatment tool is inserted into a lumen or a drug is administered thereto, thus facilitating the insertion operation and the administration operation.

[Second Example of Drug Administration]

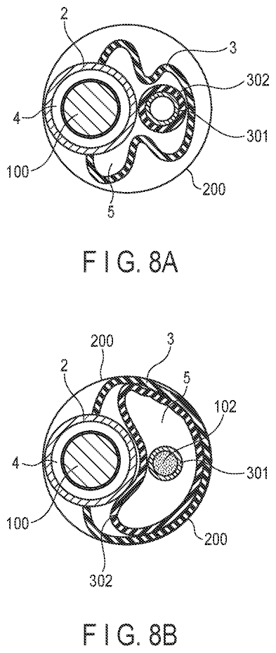

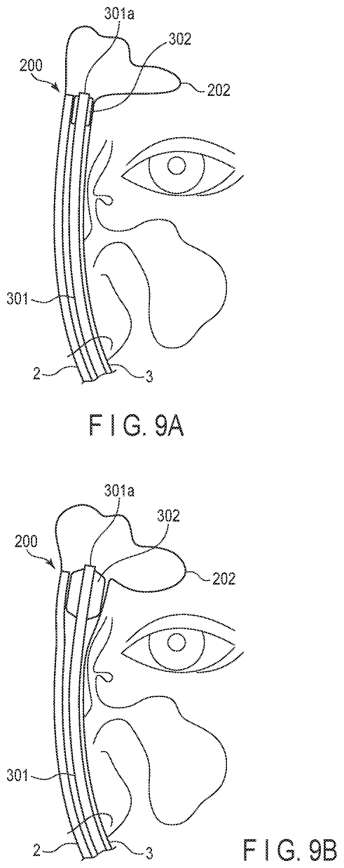

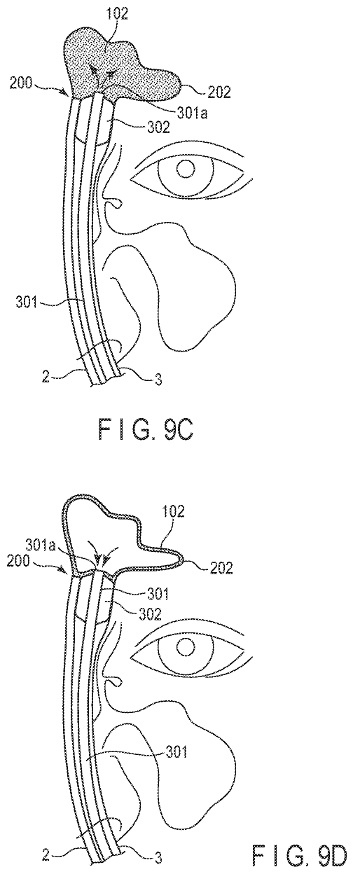

Next, a second example of drug administration to the overtube 1 using the catheter 301 including the balloon 302 according to the present embodiment will be described. FIGS. 8A and 8B are diagrams each showing a cross-sectional configuration of a state in which the catheter 301 including the balloon 302 is inserted into the overtube. FIG. 9A is a conceptual diagram showing a state in which the overtube 1 into which the catheter 301 including the balloon 302 has been inserted is inserted into the frontal sinus through the nasal passage, and FIG. 9B is a conceptual diagram showing a state in which the balloon of the catheter is inflated. FIG. 9C is a diagram showing a state in which a drug is administered into the sinus through the catheter 301, and FIG. 9D is a diagram showing a state in which the drug administered into the sinus is discharged by suction, with a requisite amount of the drug maintained.

In FIG. 5B described above, depending on the amount of the drug 102 to be administered, the unfolding of the wall portion of the secondary tube 3 caused by the expansion of the catheter 101 may not occur to an extent that fills in the ventilation hole (orifice 200) of the frontal sinus 202, thus generating a large gap between the orifice 200 and the overtube 1. When the orifice of the sinus of the paranasal sinuses is the frontal sinus 202, which is located on the lower side as viewed in the gravitational direction, there may possibly be a case where the administered drug flows out from the gap of the orifice to the nasal passage, etc., thus preventing the drug from naturally staying in the frontal sinus 202. To avoid this, an example will be described in which a drug is administered using a catheter including a balloon to fill in the gap of the orifice with the balloon.

First, the endoscope 100 is inserted into the lumen 4 of the primary tube 2 of the overtube 1 in the orifice 200 of the frontal sinus 202. Subsequently, as shown in FIG. 9A, a distal end of the overtube 1 to which the endoscope 100 is attached is inserted from the nasal cavity 201 to reach the orifice 200 of the frontal sinus 202 of the paranasal sinuses through the nasal passage, in a manner similar to FIG. 4 described above. Thereafter, a distal portion of the secondary tube 3 of the catheter 301 is introduced from the insertion port up to the distal opening 3d. The balloon 302 of the catheter 301 during the insertion is not inflated, as shown in FIG. 8A.

After that, the balloon 302 of the catheter 101 is inflated by infusing a fluid such as a gas, as shown in FIGS. 8B and 9B. In accordance with the inflation of the balloon 302, the wall portion with the folded structure of the secondary tube 3 is unfolded, and is expanded to follow the shape of the orifice 200 of the frontal sinus 202. By this unfolding, the overtube 1 is expanded in the radial direction, thus bringing the overtube 1 and the orifice 200 into close contact and reducing the gap.

Thereafter, the drug 102 is administered through the catheter 301 in such a manner that the interior of the frontal sinus 202 is filled with the drug 102 so as to apply the drug 102 to the entire inner wall surface of the frontal sinus 202, as shown in FIG. 9C. The administered drug 102 used herein has a gel-like viscosity, and adheres well to the wall surface in the sinus.

After that, the drug 102 filled into the frontal sinus 202 is discharged by suction to the outside through the catheter 301, as shown in FIG. 9D. After this discharge, the drug remains to adhere to the wall surface of the frontal sinus 202, thus allowing a requisite amount of the drug to have been applied. By thus discharging the excess amount of the drug, it is possible to prevent pain, etc. caused by the drug that remains to be filled into the sinus.

In the second example, a description has been given of the case where the treatment target is the paranasal sinuses and the position of the orifice is located on the lower side as viewed in the gravitational direction; however, the present invention can be applied to a treatment target including an orifice located on a lateral side of the body cavity or on an upper side as viewed in the gravitational direction. In this case, a catheter 301 including a catheter portion extending from a balloon 302 is used, by providing the balloon 302 at a position shifted from a distal end 301a of the catheter to the proximal side. That is, when the orifice 200 is occluded by the balloon 302 of the catheter 301, it is only required that the catheter 301 extend into the sinus, and that the distal end 301a of the catheter 301 be located in the vicinity of the bottom surface of the sinus.

[Third Example of Drug Administration]

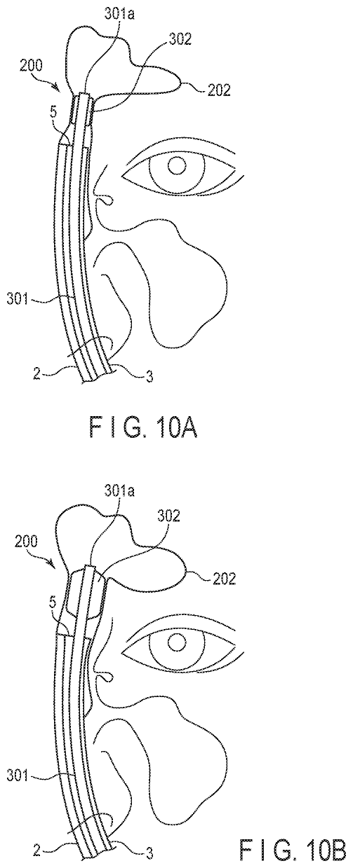

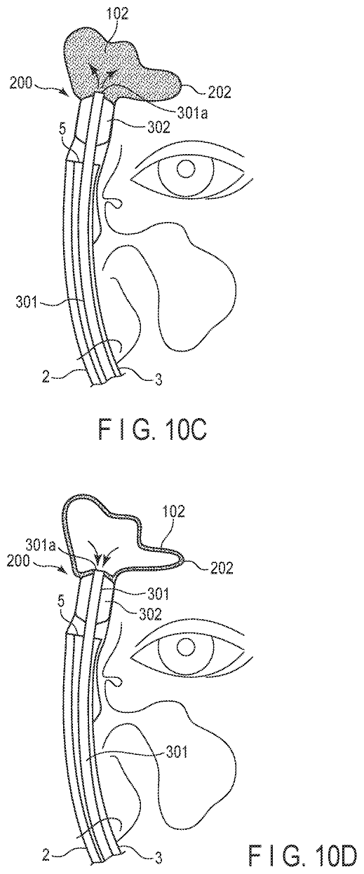

Next, a third example of drug administration using the catheter 301 including the balloon 302 which protrudes from the lumen 5 of the overtube 1 of the present embodiment will be described. FIG. 10A is a conceptual diagram showing a state in which the catheter 301 including the balloon 302 is inserted into the frontal sinus so as to protrude from the overtube 1, and FIG. 10B is a conceptual diagram showing a state in which the balloon 302 of the catheter 301 is inflated. FIG. 10C is a diagram showing a state in which a drug is administered into the sinus through the catheter 301 with the balloon 302 inflated, and FIG. 10D is a diagram showing a state in which the drug administered into the sinus is discharged by suction, with a requisite amount of the drug maintained.

In the second example described above, the balloon 302 of the catheter 301 is inflated in the lumen 5 to occlude the orifice 200 of the frontal sinus 202 together with the wall portion of the overtube 1; however, the third example is an example in which the orifice 200 of the frontal sinus 202 is occluded only by the balloon 302 to further improve the sealing property.

First, the overtube 1 is inserted through the nasal passage up to the front of the frontal sinus 202 while observation is performed with the endoscope, in a manner similar to the above-described manner. After that, as shown in FIG. 10A, the catheter 301 including the balloon 302 is introduced into the lumen 5 of the secondary tube 3, and is inserted up to the distal portion of the overtube 1. The catheter 101 is further inserted in such a manner that its distal portion protrudes from the lumen 5 in such a manner that it fills the orifice 200 of the frontal sinus 202. At this time, the folded structure of the lumen 5 is unfolded only by an amount corresponding to the diameter of the catheter 101.

After that, the balloon 302 is inflated by infusing thereinto a fluid such as a gas, as shown in FIG. 10B. At this time, the inflation is performed until there is no gap between the orifice 200 of the frontal sinus 202 and the balloon 302. Thereafter, as shown in FIG. 10C, a drug 102 having a gel-like viscosity is administered into the frontal sinus 202 through the catheter 301 until the interior of the frontal sinus 202 is filled with the drug 102. After that, as shown in FIG. 10D, the drug 102 filled into the frontal sinus 202 is discharged by suction to the outside through the catheter 301. As a result of this discharge, the drug remains to adhere to the wall surface of the frontal sinus 202, thus allowing a requisite amount of the drug to have been administered.

According to the third example, since the orifice 200 of the frontal sinus 202 is occluded only by the inflated balloon 302, the occlusion property increases, and the amount of the drug leaking from the orifice 200 during the drug administration can be further reduced. The third example is an example in which insertion is performed using the overtube 1, and the lumen is arranged to extend to the front of the orifice 200 of the frontal sinus 202. However, in a situation in which an endoscope, etc. is unnecessary, and insertion can be performed using only a catheter including a balloon up to a drug administration position without using the overtube 1, only the catheter may be inserted to occlude the orifice with the balloon. Through such occlusion, it is possible to prevent the drug from being leaked during the administration of the drug.

### Modification of First Embodiment

In the secondary tube 3 of the first embodiment described above, a wall portion formed of a smooth surface is folded in a portion with the folded structure of the distal section 3a.

As a first modification, the portion with the folded structure may be formed of a wall portion with punched holes. By punching holes in the portion with the folded structure, it is possible to reduce the weight. Since the elastic force of the wall portion is weakened when the folded structure is unfolded, the expansion is facilitated when an insertion device such as a catheter is inserted thereinto.

As a second modification, a portion with the folded structure may be formed in a mesh structure. In this case, the mesh is expanded as the folded portion is unfolded. This shortens the folded overlapping portion, and the bulge of the portion with the folded structure at the distal section 3a can be thinned out. In some cases, the overlapping portion of the wall portion with the folded structure is eliminated, causing the secondary tube 21 to stick to an outer peripheral surface of the primary tube 2.

With this second modification, it is possible to reduce the weight of the portion with the folded structure. In addition, the thickness of the overlapping portion of the portion with the folded structure is reduced, thus reducing the diameter of the overtube and facilitating the insertion operation into the treatment target. When, for example, an insertion device such as a catheter is introduced into the portion with the meshed folded structure, the overlap is unfolded as the meshed portion is expanded by a requisite amount, thus facilitating the insertion of the insertion device. Moreover, the cross-sectional area of the wall portion with a meshed structure according to the second modification, which includes the spreading of the meshed portion in addition to the unfolding of the folded portion, becomes greater than the cross-sectional area of the unfolded wall portion configured of a smooth surface according to the first embodiment. That is, assuming that the secondary tube 3 has the same cross-sectional area when unfolded, the portion with the folded structure can be made more compact.

As a third modification, small hemispherical projections are provided over a smooth surface which forms the inner surface of the wall portion with the folded structure. By providing the spherical projections to reduce the contact area with the surface of the insertion device to be inserted, it is possible to reduce the contact resistance during insertion.

The present invention is not limited to the above-described embodiment, and various modifications can be made without departing from the scope of the present invention. In addition, the embodiments may be appropriately combined and implemented, and, in this case, combined effects are obtained.

1. An overtube in which at least two lumens are aligned and which is inserted into a subject, said at least two lumens including a first lumen and a second lumen, the overtube comprising:
a first wall portion which forms an anterior side of the second lumen to receive insertion, the anterior side having a folded structure in which the distal side is folded so as to be unfoldable in a radial direction; and
a second wall portion which forms a posterior side of the second lumen to be gripped and which is more rigid than the first wall portion in the radial direction, the posterior side including an opening of the second lumen.

2. The overtube according to claim 1, further comprising:
a third wall portion which couples the first wall portion and the second wall portion in a longitudinal axis direction, which is more rigid than the first wall portion, and which is softer than the second wall portion.

3. The overtube according to claim 2, wherein a rigidity of the third wall portion smoothly changes a rigidity of an end portion of the first wall portion to a rigidity of an end portion of the second wall portion with variations.

4. The overtube according to claim 2, wherein the third wall portion is in a shape in which the folded structure is unfolded.

5. The overtube according to claim 1, wherein the first wall portion is configured in such a manner that the folded structure is unfolded by an insertion object inserted from the posterior side of the second lumen.

6. The overtube according to claim 1, wherein the wall portions are coupled in such a manner that a portion of an outer peripheral surface in a longitudinal axis direction at the wall portion forming the first lumen is shared in a longitudinal axis direction as a part of a wall portion of the second lumen, and
the first wall portion has a folded structure in which the first wall portion rises from an outer peripheral surface of the wall portion forming the first lumen, and is folded along an outer peripheral surface of a wall portion forming the first lumen.

7. The overtube according to claim 1, wherein a length of a soft portion with a folded structure of the first wall portion of the second lumen in the longitudinal axis direction is at least a length to be inserted into an insertion target.

8. The overtube according to claim 5, wherein
an endoscope is inserted into the first lumen as an insertion object, and
a catheter or a treatment tool is inserted into the second lumen as an insertion object.

9. The overtube according to claim 8, wherein a given drug is filled into the catheter in advance up to a distal end of the catheter prior to insertion of the overtube.

10. The overtube according to claim 1, wherein a breadth of a cross-section of the second lumen expands in a tapered manner toward the posterior side in the unfolded state from the posterior side.

11. The overtube according to claim 1, wherein projections are provided on an interior surface of the second lumen.

12. The overtube according to claim 1, wherein holes are punched in at least the first wall portion of the wall portions configuring the second lumen.

13. The overtube according to claim 2, wherein the third wall portion is formed using a material that is identical to a material of at least one of the first wall portion and the second wall portion and that has a thickness different from a thickness of the material of at least one of the first wall portion and the second wall portion to provide a different rigidity.

14. The overtube according to claim 2, wherein the third wall portion is formed using a material that is different from a material of the first wall portion and a material of the second wall portion to provide a different rigidity.

15. An overtube which includes at least two lumens including:
a first lumen which is formed of an elongated tubular wall portion having a flexibility and into which an endoscope is inserted; and
a tubular second lumen which is provided adjacent to the first lumen, which is formed of an elongated wall portion having a flexibility, and into which a medical device including a catheter is inserted, the overtube comprising:
a first wall portion which forms an anterior side of the second lumen to receive insertion, the anterior side having a folded structure in which the anterior side is folded so as to be unfoldable;
a second wall portion which forms a posterior side of the second lumen configured to be gripped and which is more rigid than the first wall portion in the radial direction; and
a third wall portion which couples the first wall portion and the second wall portion, which is more rigid than the first wall portion and which is softer than the second wall portion, and which has a shape in which the folded structure is unfolded in the radial direction.

16. A treatment method which uses the overtube according to claim 1, wherein
the overtube is inserted into a body cavity or a surgical portal, with the first wall portion folded, in such a manner that at least the first wall portion is arranged in a body cavity,
a treatment tool or a drug is introduced into the second lumen from a side of the second wall portion,
the treatment tool or the drug is inserted into the second lumen on a side of the first wall portion in such a manner that the first wall portion in the folded state is unfolded.

17. The treatment method according to claim 16, wherein when the treatment tool or the drug is introduced into the second lumen from a side of the second wall portion,
the second wall portion is gripped.

18. The treatment method according to claim 16, wherein the drug is filled into a catheter, and the catheter is inserted into the second lumen.

19. The treatment method according to claim 16, wherein when the treatment tool or the drug is inserted into the second lumen on a side of the first wall portion in such a manner that the folded first wall portion is unfolded, the first wall portion is unfolded in the radial direction.

20. The treatment method according to claim 16, wherein the body cavity is a nasal cavity.
